(19) United States
(12) Patent Application Publication
(10) Pub. No.: US 2026/0144607 A1
(43) Pub. Date: May 28, 2026

(54) ROBOTIC ANGIOPLASTY

(71) Applicant: BIOTRONIK AG, Buelach (CH)

(72) Inventors: Matthias Wesselmann, Ruedlingen (CH); Jeremy Wernli, Wettingen (CH); Azadeh Mehrabi, Meilen (CH); Bodo Quint, Dettighofen (DE)

(21) Appl. No.: 19/123,285

(22) PCT Filed: Dec. 8, 2023
(86) PCT No.: PCT/EP2023/084858

§ 371 Date: Apr. 22, 2025

(30) Foreign Application Priority Data
Dec. 12, 2022 (EP) 22212725.0

(51) Int. Cl. A61B 34/00; A61B 34/20; A61B 34/30; A61M 25/01

(52) U.S. Cl. CPC A61B 34/71; A61B 2034/2065; A61B 2034/301; A61M 25/0113

(57) ABSTRACT

A catheter system includes a rolling membrane guiding catheter and a docking unit. The docking unit is configured to automatically insert a plurality of devices into the rolling membrane guiding catheter.

### PRIORITY CLAIM

This application is a 35 U.S.C. 371 US National Phase and claims priority under 35 U.S.C. § 119, 35 U.S.C. 365(b) and all applicable statutes and treaties from prior PCT Application PCT/EP 2023/084858, which was filed Dec. 8, 2023, which application claimed priority from EP Application 22212725.0, which was filed Dec. 12, 2022.

### FIELD OF THE INVENTION

A field of the invention concerns catheter devices, and particularly rolling membrane guiding catheters.

### BACKGROUND

Medical treatment of a stenosis via percutaneous coronary intervention (PCI) can involve the insertion of a catheter into a blood vessel at the position of the stenosis. Often more than one member is introduced into the respective blood vessel to perform the different steps of the treatment. In such procedures, catheters can be introduced as a guiding catheter through which further members/tools can be introduced into the blood vessel to the position of the treatment site, e.g., a stenosis to perform their respective function there in a safe way without directly interacting with the vessel walls during their insertion. It can be especially advantageous to provide systems for automated control of the guiding catheter and/or other members introduced into the blood vessels. However, there are issues that require improvement in such automated catheter systems.

Complete angioplasty procedures based on current rapid exchange and/or over the wire design are very hard to automate due to guide wire insertion into guide wire lumen. Therefore, the insertion of new members, tools and/or catheters into a potentially already introduced guiding catheter requires manual labor by a member of the medical staff.

Further, for hygiene reasons, any portion of such a catheter system that can have been in contact with the catheter during the treatment must be disposed afterwards and replaced by sterile replacements.

Using standard guiding catheters and/or introducer sheaths, there is further no guarantee that the distal tip of the guiding catheter and/or introducer sheath holds its position when a guided catheter runs into an obstacle and pushes the guiding catheter and/or introducer sheath back. This is one of the reasons why current catheter placement struggles with precision.

Generally, there are various drawbacks with non-automated catheter systems which include, e.g., that balloon choice (diameter and length) based on angiography is prone to error and generally, procedures, especially those to remove and/or open a chronic total occlusion, are too time-consuming.

### SUMMARY OF THE INVENTION

A preferred embodiment is a catheter system that includes a rolling membrane guiding catheter. A docking unit is configured to provide automatic insertion of a plurality of devices into the rolling membrane guiding catheter. The plurality of devices can include at least one of the following: a guidewire, a dilator catheter, a balloon catheter, a stent, a further rolling membrane catheter, a biopsy tool, an endoscope, an intravascular ultrasound, IVUS, tool, a fractional flow reserve, FFR, tool, and/or an optical coherence tomography, OCT, tool.

### BRIEF DESCRIPTION OF THE DRAWINGS

The Figures of the present disclosure show the following:

FIG. 1 Exemplary embodiment of a catheter system including a guiding catheter and multiple further catheters.

FIG. 2 Further exemplary embodiment of a catheter system including a guiding catheter and multiple further catheters.

FIG. 3 Exemplary embodiment of a rolling membrane catheter as a guiding catheter of the catheter system.

FIG. 4 Schematic representation of exemplary configuration of a catheter system.

FIGS. 5A-G Schematic representation of introduction of the rolling membrane guiding catheter into cardiovascular system, guidewire rotation, advancement of the rolling membrane guiding catheter and guidewire to and beyond the treatment site, dilation of an exemplary stenosis by a dilator catheter, of the retracted dilator catheter and guidewire, and of the eversion of the rolling membrane of the rolling membrane guiding catheter to widen the stenosis.

FIGS. 6A-6C Exemplary arrangements of two transport rollers for translationally movement and/or rotation of a device

### DETAILED DESCRIPTION OF THE PREFERRED EMBODIMENTS

A preferred embodiment is a catheter system including a docking unit for automated insertion of a plurality of devices into a rolling membrane guiding catheter and respective methods and computer programs.

According to one aspect of the invention, a catheter system can be provided including a rolling membrane guiding catheter and a docking unit. The docking unit can be provided for automatically inserting a plurality of devices into the rolling membrane guiding catheter.

The combination of a rolling membrane catheter with the docking unit enables various benefits: First, the insertion of the rolling membrane catheter can reduce the risk of harming the vessel walls and provide a user-friendly and safe tool, relaxing the requirements as to the precision of automatization. Second, using the rolling membrane catheter as a guiding catheter increases the safety during the insertion of any other device(s) into the lumen to be treated as the rolling membrane catheter can pose a protective layer between the at least one device and the vessel wall. In contrast to other catheters, the flexibility of the rolling membrane can allow further operations performed by the at least one device which can rely on an at least partly deformable guiding catheter to treat the vessel walls, e.g., widening a stenosis. Further, a docking unit can pose the advantage that any device can be inserted. This can be particularly advantageous as during a treatment, it might not be obvious from the beginning which devices can be required. Therefore, the docking unit can increase the flexibility in choosing the devices that can be available during the treatment. And a rolling membrane catheter can offer a particularly beneficial hub for automatic insertion due to its flexibility and additional degrees of freedom such as being (partially) rolled-in or rolled/out and/or (partially) inflated or deflated.

In some examples, the rolling membrane catheter can include a rolling membrane, an outer shaft, and an inner shaft, wherein the rolling membrane can be connected to a distal end of the outer shaft and a distal end of the inner shaft. The rolling membrane of the rolling membrane catheter can be everted in the lumen to be treated to and beyond the treatment site by providing a pressure to the rolling membrane volume, limited by the inner shaft, the outer shaft, and the rolling membrane, and simultaneously moving the inner shaft distally relative to the outer shaft.

In an example, the docking unit can be connected to the inner shaft to allow any device introduced through the docking unit to unimpededly advance through the inner shaft to the treatment site with minimal or no interaction with the vessel walls, if intended. The docking unit may, in some examples, receive a plurality of devices at the same time, repeatedly, and/or consecutively. This provides an extraordinarily high degree of freedom in tailoring the treatment to the patient and/or the (lack of) success in previous treatment steps. This can significantly increase the prospect of successful treatment and reduce the required treatment time and the danger of damage to the vessel walls and the devices used during the treatment.

In some examples, the docking unit is configured as a funnel. The docking unit can include an exit opening and an entrance opening, wherein the exit opening is smaller than the entrance opening. For example, the exit opening and the entrance opening are centralized around a longitudinal axis of the docking unit, and the cross sectional area of the exit opening with respect to the longitudinal axis is smaller than the cross sectional area of the entrance opening with respect to the longitudinal axis. Thus, the docking unit can be connected to (the inner shaft of) the rolling membrane guiding catheter via the exit opening to allow at least one of the devices introduced through the docking unit via the entrance opening to be introduced to the (the inner shaft of) the rolling membrane guiding catheter.

In some examples, the docking unit includes a wall between the exit opening and the entrance opening. The wall is inclined with respect to the longitudinal axis, preferably by less than 45°, and more preferably by less than 30°. This allows that the devices can be safely funneled through the docking unit into (the inner shaft of) the rolling membrane guiding catheter with ease.

In some further examples, the docking unit includes guide channels for guiding the devices from the entrance opening to the exit opening. Preferably, each guide channel is configured to guide a single device. For example, each guide channel can be configured as a tunnel including a tunnel wall. This allows that the devices can be safely introduced to (the inner shaft of) the rolling membrane guiding catheter as the devices do not hinder each other's movement within the docking unit due to separation by the guide channels.

In an example, each guide channel includes suitable connectors to provide hydraulic pressure and/or electricity. Further, each guide channel can include a component to read out data from the device inserted to the guide channel (e.g. device identification data or data measured by the device).

In an example, the insertion can be automated such that no staff is required to be present to, e.g., exchange devices and/or insert new devices. This can decrease the cost per treatment, risks associated with close contact with potentially contagious patients, the risk of erroneous operation by the staff, etc. In some examples, the plurality of devices to be inserted through the docking unit of the catheter system of claim 1 includes at least one of the following: a guidewire, a dilator catheter, a balloon catheter, a stent, a (further rolling membrane) catheter, a biopsy tool, an endoscope, an intravascular ultrasound, IVUS, tool, a fractional flow reserve, FFR, tool, and/or an optical coherence tomography, OCT, tool.

For example, a guidewire can be inserted via the docking unit. This guidewire can be inserted as a first guidewire or additionally to at least one guidewire, which can already be placed within the rolling membrane guiding catheter. A second guidewire can for example be inserted to guide a further device along the second guidewire into a different direction than the first guidewire, e.g., to place a blocking component to block a branch of the cardiovascular system that should not be entered by any other catheter and/or device.

In a further example, a dilator catheter can be inserted via the docking unit. The dilator catheter can be used to expand the lumen to be treated radially outward, which can be achieved by an expanding dilator catheter and/or a conically shaped tip of the dilator catheter to effectively increase the dilator radius at the treatments site when the dilator catheter is moved distally beyond the treatment site. The dilator catheter can be made from any soft and/or at least partially compressible material to prevent damaging the vessel walls.

In some examples, the at least one device inserted through the docking unit can be a balloon catheter. The balloon catheter can include a shaft with a balloon connected to a distal end region of the balloon catheter shaft. The balloon can first be advanced through the rolling membrane guiding catheter to the treatment site and then caused to expand by providing pressure by a balloon fluid unit to the balloon, e.g., via the balloon catheter shaft. This expansion can then apply a radial force to the vessel wall to widen a stenosis.

The device can also for example be a stent. Such stent can include a grid-like structure configured to expand at the treatment site to permanently stabilize and widen a stenosis. The stent can furthermore include a medical agent continuously delivered to the surrounding tissue to inhibit, e.g., ingrowth, inflammation, and/or cicatrization. In some examples, a (further rolling membrane) catheter can be provided and inserted into the rolling membrane guiding catheter. The further catheter can have a smaller diameter than the rolling membrane guiding catheter.

The biopsy tool can be any component configured for extraction of sample tissue for evaluation. This can allow to determine for example the texture of the stenosis to be treated to choose an appropriate treatment strategy.

The endoscope can be any inspection instrument composed of, e.g., an image sensor, an optical lens, a light source and/or a mechanical device. It can be configured to image lesions that cannot be detected by for example X-ray-based imaging methods.

The IVUS tool can be any ultrasound-based device for invasive imaging to be introduced into the cardiovascular system of the patient.

The FFR tool can be any diagnostic device configured to measure, e.g., pressure differences across a stenosis.

The OCT tool can be used to image coronary arteries to visualize vessel wall lumen morphology and microstructure at a high resolution. Typically, ca. 1 mm-diameter catheters based on fiber-optics can be used. These can access the lumen to be treated via the rolling membrane guiding catheter.

Any further devices, which are not described herein, can be introduced through the docking unit.

In some examples, the catheter system can further include at least one fluid unit for determining a fluid state of the rolling membrane and/or at least one of the plurality of devices. Determining the fluid state of any member can provide a useful control over various devices which is compatible with the required flexibility of the used catheters and/or devices.

The fluid state can relate for example to a fluid pressure and/or a fluid flow. The fluid can be any liquid fluid, gaseous fluid, and/or any mixture thereof. For example, the fluid unit can be a pressure pump for providing a fluid pressure and/or a fluid flow and/or a fluid sensor for sensing any parameter associated with the fluid state.

The at least one fluid unit can contribute to and/or induce different modes in which, e.g., the rolling membrane guiding catheter can be used. For example, a constant fluid pressure and/or constant fluid flow can be provided to the rolling membrane during eversion and/or retraction of the rolling membrane to continuously keep the rolling membrane in an at least partly inflated state facilitating the rolling motion of the rolling membrane. In some examples closed-loop configurations can be implemented in which a fluid state is induced and simultaneously sensed.

In an example, the catheter system can further include at least one motion unit configured to determine a motion state of the rolling membrane guiding catheter, the rolling membrane, and/or at least one of the plurality of devices. The at least one motion unit can provide the possibility to determine the motion state of any component accurately, possibly with a higher accuracy than a health professional might achieve. This can increase success chances of the treatment and safety.

Herein, the term “determine” can relate to initiating a respective state via, e.g., an actuator and/or sensing a respective state, e.g., via a sensor.

The motion state can relate, e.g., to the absolute and/or relative position, velocity, and/or acceleration of any catheter, device, and/or member thereof. For example, the motion unit can be a motion actuator for providing a motion state and/or a motion sensor for sensing any parameter associated with the motion state of any component described herein. Further, the motion unit can provide electricity to the respective member it is operably coupled to.

The at least one motion unit can sense and/or induce the motion of at least one of the used catheters, devices, and/or member thereof in different modes described herein. In some examples closed-loop configurations can be implemented in which a motion state is induced and sensed simultaneously.

For example, in a “preloaded” mode, the rolling membrane of the rolling membrane guiding catheter can transport less-performant products to and through a lesion extending their range, reducing damage to the vessel wall.

In a further example, the at least one motion unit of the catheter system is configured to determine a translational component of the motion state and/or a rotational component of the motion state. This can yield a necessary control over the catheters and/or devices to accurately guide them through the cardiovascular system to the treatment site. This can provide the possibility to do so without the need of manual intervention.

The translational component and the rotational component of the motion state can be fully isolated, e.g., they can be controlled independently from each other. For example, two motion units can be provided that can rotated relative to each other by an angle of 90°. In such exemplary configuration, one motion unit can be oriented in parallel to the length axis of the controlled device and/or catheter to determine the translational component of the motion state of the controlled device and/or catheter and the second motion unit can be oriented such that it determines the rotational component of the motion state of the controlled device and/or catheter, e.g. perpendicularly to the length axis. The translational and rotational components may, however, also be coupled, e.g., both can be controlled by a motion unit that is oriented neither in a 0°, nor in a 90° angle relative to the length axis of the controlled device and/or catheter such that the same motion unit can induce a rotation and a translation at the same time.

For example, the guidewire can have a bent tip which can be rotated towards the chosen path at any bifurcation within the cardiovascular system to advance into the intended lumen via translational advancement. Further any other device can be operated via translational and/or rotational operations to fulfil the respective function. The increased degree of freedom in operating the different catheters, devices and/or members thereof can increase flexibility, accuracy, and success chances of the treatment.

For example, both components can be relevant for both, safe and accurate advancement and/or retraction of the respective catheter, device and/or members thereof.

In some examples, the motion unit includes a transport roller which is configured to rotate around a rotational axis. The transport roller is configured to move one of the plurality of devices into and out of the rolling membrane guiding catheter and/or the docking unit and/or to rotate one of the plurality of devices (i.e., controlled device) with respect to the rolling membrane guiding catheter and/or the docking unit. If the rotational axis is perpendicular to the length axis of the controlled device, the transport roller is configured to translationally move the controlled device. If the rotational axis is parallel to the length axis of the controlled device, the transport roller is configured to rotate the controlled device. If the rotational axis is neither parallel nor perpendicular to the length axis of the device (i.e., the rotational axis and the length axis of the controlled device are tilted at an angle larger than 0° and smaller than 90° to each other), the transport roller is configured to translationally move and rotate the controlled device.

In a further example, the transport roller is configured to rotate around a third rotational axis, wherein the third rotational axis is perpendicular to the first rotational axis. Rotating the transport roller around the third rotational axis can determine whether the transport roller is configured to translationally move the controlled device and/or rotate the controlled device. If the third rotational axis is perpendicular to the length axis of the controlled device, rotating the transport roller around the third rotational axis determines the angle between the rotational axis of the transport roller and the length axis of the controlled device. Thus, the ratio of the translational movement and the rotation of the controlled device can be adjusted: an angle of 0° between the rotational axis and the length axis of the controlled device (i.e., rotational axis is parallel to the length axis) enables a rotation of the controlled device, whereas an angle of 90° between the rotational axis and the length axis of the controlled device (i.e., rotational axis is perpendicular to the length axis) enables a translational movement of the controlled device along the length axis.

In some examples, the at least one motion unit includes a first transport roller configured to rotate around a first rotational axis and a second transport roller configured to rotate around a second rotational axis, wherein the first transport roller and the second transport roller are arranged to form a gap between each other along a third axis, and the first rotation axis lies in a first plane and the second rotation axis lies in a second plane, wherein the third axis is orthogonal to the first plane and to the second plane.

In some examples, the gap is configured such that the one of the plurality of devices can be arranged within the gap in a force-fitting manner along the third axis.

Accordingly, the one of the plurality of devices can be translationally moved and/or rotated. Thus, the first transport roller and the second transport roller can move one of the plurality of devices into and out of the rolling membrane guiding catheter and/or the docking unit and/or to rotate one of the plurality of devices with respect to the rolling membrane guiding catheter and/or the docking unit.

In some examples, the first transport roller and the second transport roller are configured to be movable relative to each other along the third axis. Accordingly, a device can be placed in a force-fitting manner along the third axis between the first and the second transport roller.

In some examples, the first rotational axis and the second rotational axis are parallel. That is, the first transport roller and the second transport roller can translationally move the controlled device along a length axis of the controlled device if the length axis is orthogonal to the first rotational axis and the first transport roller rotates in the opposite direction around the first rotational axis to the second transport roller around the second rotational axis. Alternatively, the first transport roller and the second transport roller rotate the controlled device around a length axis of the controlled device if the length axis is parallel to the first rotational axis and the first transport roller and the second transport roller rotate in the same direction around the first rotation axis, respectively, second rotation axis.

In some examples, the projection of the first rotational axis to a plane orthogonal to the third axis and the projection of the second rotational axis to the plane orthogonal to the third axis enclose an angle between 0° and 180°. That is, the first transport roller and the second transport roller translationally move the controlled device (i.e., one of the plurality of devices) along the length axis of the controlled device and rotate the controlled device around the length axis. In case the first rotational axis is rotated at the same speed as the second rotational axis, projections of co-rotating ends of the first rotational axis and the second rotational axis (i.e., ends that rotate in the same direction) into a plane orthogonal to the third axis are preferably mirror-symmetrical to the length axis of the controlled device to allow for a balanced and stable translation and rotation of the controlled device.

In some examples, the first transport roller is configured to rotate around a fourth rotational axis and the second transport roller is configured to rotate around a fifth rotational axis, wherein the fourth rotational axis is parallel or identical to the third axis, and the fifth rotational axis is parallel or identical to the third axis. By rotating the first and the second transport roller independently around the third axis or an axis parallel to the third axis, the arrangement of the first and second transport roller can be adjusted so that the one of the plurality of devices can be moved in different ways: translation of the device only, rotation of the device only or translation and rotation.

In some examples, the first transport roller and/or the second transport roller include a material (e.g., silicone) and/or surface structure (e.g., knobs and/or grooves) which is configured to enhance friction between the first and/or second transport roller and the one of the plurality of devices.

In some examples, the at least one motion unit of the catheter system can be configured to be equipped with at least one exchangeable and disposable transport belt. This can have the advantage that a sterile system can be provided reliant on only minor replacements which can reduce costs and manual labor. Providing sterile work environments is typically required by medical standards and therefore essential to achieve.

In one example, the exchangeable and disposable transport belt is placed around a transport roller. The exchangeable and disposable transport belt can include a material (e.g., silicone) and/or surface structure (e.g., knobs and/or grooves) which is configured to enhance friction between the transport roller and the one of the plurality of devices.

In addition or alternative to the transport roller, the at least one motion unit can include conveyor belts for translationally moving and/or rotating one of the plurality of devices. The conveyor belts can be arranged as the transport rollers. A conveyor belt can include an exchangeable and disposable transport belt including a material (e.g., silicone) and/or surface structure (e.g., knobs and/or grooves) which is configured to enhance friction between the transport roller and the one of the plurality of devices.

The catheter system can for example further include at least one component to identify at least one of the plurality of devices. This can reduce the need for human intervention and can even render it obsolete. This can increase efficiency and flexibility allowing to introduce the needed device(s).

For example, the component to identify can identify the at least one device of the plurality of devices by the shape of at least a part of the device to be introduced, a magnetic label, an RFID tag, any visual code like text code, number code, symbol code, a QR code, barcode, and/or color code, and/or FTIR-code. When the respective device is identified, it can be inserted via the docking unit by at least one motion unit. Otherwise, operation can be halted, such as to increase safety.

For examples, the component to identify can include at least one optical and/or mechanical sensor adapted to the respective used code, e.g., a barcode scanner.

In some examples, the docking unit of the catheter system can include a pressure-tight seal. This can have the advantage to prevent any contamination of the cardiovascular system of the patient while simultaneously allowing devices to be inserted via the docking unit and to provide a pressure to the inside of the catheter, e.g., to the inner shaft of the rolling membrane guiding catheter.

In a further example, the catheter system includes a supply line which is fluidly connected to the inner shaft of the rolling membrane guiding catheter. In some examples, the docking unit can include a port which fluidly connects the supply line with the inner shaft of the rolling membrane guiding catheter. The supply line can be configured to carry a contrast agent. In some examples, either the docking unit or the supply line can include a hemostasis valve for (automized) opening or closing of the port, respectively, the supply line.

According to these embodiments, a fluid, e.g. a contrast agent, can be lead into the inner shaft of the rolling membrane guiding catheter. The distal end of the rolling membrane guiding catheter can be used to seal the inner shaft of the rolling membrane guiding catheter against the vessel.

In some examples, the catheter system can further include an imaging device and/or a device for image evaluation. The combination with an imaging device can further increase the degree of automatization such that the required human input can be reduced such that image data can be directly processed by the catheter system. This can increase treatment accuracy, reliability, and speed.

The image data can include images of the treatment site and can be acquired before the treatment and/or during the treatment, e.g. via (active) tracking of the vessels and/or the position of the used catheters and/or tools. The image evaluation device can further be configured to receive as-acquired image data from the imaging device and/or image data acquired, e.g., by a separate imaging device, in a concurrent and/or a previous imaging session.

The imaging device can in some examples be based at least in part on (contrast agent-based) X-ray angiography, computed tomography, optical coherence tomography, intravascular ultrasound imaging, and/or any combination thereof. The device for image evaluation can be adapted to evaluate any image data acquired by the imaging methods named herein and/or any other image data.

In an example, the catheter system can further be configured to determine the fluid and/or motion state of at least one member of the catheter system at least in part based on image data. This can have the further advantage to adjust the treatment to the exact geometry and/or texture of the treatment site to improve success chances and safety. Herein, a member can be a catheter, a device, and/or any subunit thereof.

Besides, the image data, the fluid state and/or motion state can also provide information related to the geometry and/or texture of, e.g., a stenosis when for example a balloon catheter can be used to palpate the lumen to be treated. The according feedback from the motion unit and/or fluid unit of the palpating device can be evaluated by the control unit to extract information on the stenosis.

The image data can be provided by any imaging component and/or imaging method described herein. For example, the chiseling motion of the guidewire can be adjusted to the stenosis to be opened in terms of frequency, amplitude, duration, choice of the guidewire tip, and/or position relative to the stenosis. This can increase safety and reduce the time required for the procedure.

In a further example, the catheter system can further include a control unit. The control unit can be operably coupled to the catheter system for automatic and/or synchronized control of at least one parameter associated with the motion state and/or fluid state of the rolling membrane guiding catheter and/or at least one of the plurality of devices. Any functionality described in reference to the control unit can as well be implemented in the at least one fluid unit and/or the at least one motion unit and vice versa. This can increase the possibility of automatization, e.g., of action sequences to be induced via the control unit in a faster, more precise, and safer way than a user might be able to.

Any coupling described herein can involve time delays due to signal propagation time and/or processing time, and synchronization of one (sub-)unit with any other (sub-)unit can therefore occur with an according time delay. The signaling can be direct and/or indirect, e.g., via the control unit (such as a microprocessor, a microcontroller, etc.) or provided separately.

The control unit can be suited to implement at least one of the following functionalities. The catheter system can be able to prepare all devices (e.g. via the evacuation of a balloon lumen and/or flushing), to check the status of devices (e.g. fluid state, motion state, calibration status, etc.), to perform the respective procedure as described herein, to monitor critical parameters (e.g. time during which for example the rolling membrane was in an inflated state, fluid pressure provided to the rolling membrane, etc.), and, if unexpected situations occur (deviations like reaching a rolling membrane burst pressure and/or encountering a fully closed stenosis) to propose modifications (e.g. change to high pressure balloon, scoring (moving a catheter with e.g. micro-blades through the plaque of a stenosis to relax the plaque to prepare for example the use of a balloon), applying lithotripsy by sending focused ultrasonic energy or shock waves directly to the surrounding, etc.).

The control unit can be an integral part connected to the catheter system or a remote part configured to communicate with the at least one fluid unit and/or the at least one motion unit. This communication can for example include sending instructions based, e.g., at least in part on at least one parameter associated with the fluid state and/or motion state of at least one member of the catheter system and/or it can include receiving feedback/signaling based, e.g., at least in part on at least one parameter associated with the fluid state and/or motion state of at least one member of the catheter system.

This can for example be implemented in a way that one parameter is measured by at least one fluid unit and/or motion unit and in adjustment to that parameter, at least one other parameter can be induced as instructed by the control unit. In a closed-loop configuration, this can relate to the same parameter such that one parameter is continuously induced and monitored to be corrected if necessary.

Further such coupling of the units of the catheter system for synchronized operation allows to initiate the catheter system to perform (pre-determined) modes, some of which are described herein as examples. Any mode described herein can for example be executed automatically in response to a single command to initiate the respective mode. It can further be stopped automatically, e.g., after a predetermined time or in response to at least one parameter measured by an internal and/or external sensor of the catheter system, or through a further command instructing the system to stop the respective mode.

In an exemplary constant fluid pressure mode, the control unit can keep the fluid pressure inside the rolling membrane at the designated level for eversion and reversion of the rolling membrane. The movement of the distal end of the rolling membrane can be controlled by moving the inner shaft of the rolling membrane catheter proximally, e.g., via the respective motion unit. A controlled fluid pressure level can be achieved, e.g., by a sufficiently large fluid container connected to the hydraulic lumen. Further, an electronic closed loop can monitor the fluid pressure level and correct it via the fluid unit, including for example a motorized pump. In some examples, the inner shaft motion can be adjusted to increase or decrease the fluid pressure inside the rolling membrane volume.

For example, in a constant fluid flow mode, the control unit can control the fluid flow into the rolling membrane volume to be constant. The fluid unit connected to the rolling membrane volume can push hydraulic fluid into the lumen at a certain constant rate until a target volume or the maximum allowable fluid pressure is reached. The fluid pressure can be monitored simultaneously. When the maximum allowed fluid pressure level is reached, the control unit can stop the pump to avoid damage to the rolling membrane. In a further example, the fluid pressure level can be controlled via the movement of the inner shaft of the rolling membrane catheter. Advancing it faster than required for the respective fluid flow can lower the fluid pressure. Advancing it more slowly can increase it. For a simpler solution, a simple mechanical apparatus can be used which can include an inflation pump with an integrated gas pressure reservoir. For safety reasons, the fluid can be contained inside a pouch that can be opened to the rolling membrane volume. This can for example be done via a valve to achieve a rather constant fluid pressure by expanding the fluid when moving the membrane. For applying higher pressures to the rolling membrane, the valve can be closed. The reversion of the rolling membrane can be performed at a lowered constant fluid pressure level while simultaneously retracting the inner shaft of the rolling membrane catheter.

In a further exemplary mode, the rolling membrane can be anchored to the vessel wall. If the rolling membrane diameter is smaller than the vessel diameter, the rolling membrane can be forced to take a spiral form that presses against the vessel wall. The rolling membrane can take a spiral form when the inner shaft is under tension. The degree of spirality can be increased by choosing a fluid pressure level and hindering the inner shaft, at least in part, to move. The control unit can control the shape of the rolling membrane by applying higher hydraulic fluid pressures to the rolling membrane than necessary for eversion. Further, the control unit can push the guidewire to assist the crossing of the rolling membrane. The guidewire movement can be used to pull the very distal end of the everting rolling membrane in the proximal direction. Guidewire and rolling membrane can therefore be designed to have a high frictional force between them.

In an exemplary “suction bell” mode, the control unit can control the generation of controlled hydraulic pulses by a rolling membrane that is “supercharged” by pulling on the guidewire and the sudden, but controlled release of it. An inflated rolling membrane that can be larger in diameter than the vessel can be anchored to the vessel wall before the narrowing. The control unit can then pull on the proximal end of the inner shaft, “charging” the rolling membrane like a bow. Releasing the inner shaft can cause a sudden hydraulic fluid pressure peak in the vessel directed to the distal obstruction/treatment site.

In an exemplary chiseling mode, the control unit can increase the effectiveness of boring a tunnel through an obstruction by concentrating the hydraulic force of a large balloon and/or rolling membrane onto a guidewire tip. The inner shaft of the rolling membrane catheter can allow to transport sharp tools without hurting the vessel. The inflated rolling membrane can be larger in diameter than the vessel and can be anchored to the vessel wall before the narrowing. The guidewire can have a stiff tip and can be advanced through inside of the rolling membrane guiding catheter towards the obstruction. The hydraulic fluid pressure inside the rolling membrane volume can be lowered until the guide wire can be moved inside the rolling membrane catheter towards the distal end of the rolling membrane and against the obstruction in the vessel. Then, the control unit can increase the hydraulic fluid pressure provided to the rolling membrane volume again to increase friction to the guide wire. The control unit can instruct the respective motion unit to pull on the proximal end of the guidewire. Due to the friction between the guidewire and the inflated and not yet everted rolling membrane, the membrane can be “charged” like a bow. Releasing the guidewire, e.g., allowing for movement along a predetermined distance, can cause a sudden acceleration and movement of the guidewire tip against the obstruction. Repeating this motion can cause a chiseling motion of the guidewire. The rolling membrane can follow the guidewire into the as-created opening and can further open a crack by applying a hydraulic force to the vessel walls.

For example, a high-pressure inflation mode can include the control unit observing how far is the rolling membrane is everted. When it can only be half everted, a high-pressure balloon could be place inside creating an ultra-high-pressure balloon. This assembly of a triple walled balloon at the treatment site can allow for a combination of ease of crossing, ultra-low profile and ultra-high pressure.

For example, in a large diameter dottering mode, a combination of an inner short balloon catheter and a larger, longer rolling membrane can allow to shift and distribute deformable fatty plaques more evenly. The rolling membrane and inner balloon can be placed inside the obstruction by the respective at least one fluid and/or motion units or manually. The inner balloon can be inflated by applying a pressure approaching the rolling membrane burst pressure. The control unit (or surgeon) can move the inflated balloon inside the inflated rolling membrane back and/or forth to model the lumen. This can provide the advantage of distributing fatty plaque more evenly. The catheter system can be adapted to host the required devices and automatically control/synchronize them.

For example, once any mode described herein can have at least partially opened an obstruction, specialized catheters like, e. g,, high pressure balloons, scoring, shockwave, IVUS, OCT, and/or FFR catheters, can be inserted through the inner shaft of the rolling membrane guiding catheter to and through the lesion that can resist further dilation. After positioning the respective device. The rolling membrane guiding catheter can be deflated and pulled back. The devices to be inserted can be stored in corresponding bays in proximity of the docking unit to be available during the procedure and be used as needed.

In some examples, the catheter system can further include a user interface. The control unit can further be configured to control the at least one parameter associated with the motion state and/or fluid state of the rolling membrane guiding catheter and/or the at least one device at least in part based on the user input. This can have the advantage that patient treatment can be controlled remotely from a different room and/or a different health institution. This can reduce the amount of manual labor and increase safety, e.g., when treating contagious patients. It can further allow the best-suited health professional to perform the treatment independent from the geographical location where the treatment is performed. However, the user interface can also be local, e.g. allowing initiating control by a nearby operator.

In an exemplary treatment, the catheter system can already be prepared based on, e.g., a CT scan analysis. Thus, the lesions to be treated are already known and an intervention strategy can be clear. The necessary devices for the percutaneous transluminal intervention can be provided to the catheter system by a member of the medical staff into the corresponding bays of the catheter system. This can include plugging in and connecting all luers/connections. In an exemplary treatment, the used catheters and devices can include a rolling membrane guiding catheter and guide wire, a specialized dilator for either accessing a right coronary artery or a left coronary artery, an optional high-pressure balloon for pre-dilatation, optional stent(s), and/or an optional post-dilatation balloon. The patient can be prepared with femoral or radial access for the catheter system. The catheter system can scan the RFID readers or barcode of catheters in the storage bays via the identification and can check each device with the PCI plan. It can also automatically vacuumize and flush the lumen of catheters. All sensors can be checked or calibrated if necessary. The staff can finally attach manually the catheter system to the introducer sheath or guiding catheter. For example, from this point, the catheter system can be controlled by a health professional via the (remote) user interface. This can occur from a different room and/or a different health institution. For example, using fly-by-wire principles, the catheter system can execute commands of the health professional entered via the user interface. These commands can instruct a treatment as described herein:

The rolling membrane guiding catheter (or a rolling membrane guiding catheter with a dilator placed inside the rolling membrane guiding catheter) and the guidewire can be pushed together by the respective motion unit(s) towards the lumen to be treated. By rotating and pushing, the guidewire tip can advance into the target lumen. The rolling membrane can be inflated for the first time following the guidewire into the (coronary) artery stem to be treated in an everting motion. Once the rolling membrane can have entered the artery, it can be deflated, and the guidewire can be retracted into the inner shaft of the rolling membrane guiding catheter. The guidewire can be controlled to remain stationary during the everting motion of the rolling membrane of the rolling membrane guiding catheter. Any other device to be introduced can be controlled and instructed accordingly.

In some examples, the catheter system can further include a primary platform and a secondary platform. The primary platform can include an introducer sheath into the patient. Further, the secondary platform can include the docking unit and can be configured to be movable relative to the primary platform.

For example, the catheter system can include two or more platforms. A primary platform can act as a connector between the patient and a secondary platform including one or more, or essentially most or even all of the catheters, devices, motion units, and/or fluid units described herein. The primary platform can be connected to the patient via an introducer sheath configured to receive the rolling membrane guiding catheter and/or any other device to be introduced into the patient. Further, the exemplary primary platform can be configured to move the whole secondary platform, onto which the respective components can be mounted, relative to the patient. At the connection between the primary platform and the secondary platform, there can be a connector with a variable length, e.g., with a telescopic structure. Due to its variable length, the (telescopic) connector can be suitable to connect the secondary platform and the introducer sheath at any time, for example when the rolling membrane guiding catheter is not yet introduced, when the guiding catheter is fully introduced into the patient, and/or at any point in between these two states, e.g. irrespective from the relative position of primary and secondary platforms.

Further, the connector can function as a motion unit determining the motion state of the outer shaft of the rolling membrane guiding catheter. The connector can further be configured to reduce its length when it moves forward to introduce, e.g., the outer shaft of the rolling membrane guiding catheter into the patient. During that insertion, the whole secondary platform can be moved with the outer shaft towards the patient and relative to the primary platform. The secondary platform can include for example, at least one motion unit operably coupled to the inner shaft of the rolling membrane guiding catheter and/or the plurality of devices. It can further include at least one fluid unit configured to provide a fluid pressure to the rolling membrane of the rolling membrane guiding catheter and/or the plurality of devices. It can further include the docking unit and storage bays for storing devices that can be required during the treatment and that can be introduced through the docking unit. Other exemplary primary and secondary platforms can include any other component described herein and functions described in reference to one platform can be implemented in the other one as well.

The secondary platform can be supported by the primary platform, e.g., such that the primary platform is larger than the secondary platform and the secondary platform can be mounted in a movable manner onto the primary platform allowing for motion of the secondary platform on the primary platform. This can yield a closed and/or stable system with yet high flexibility.

According to another aspect of the invention, a computer program including instructions can be provided. The program, when executed, can cause the catheter system to automatically couple the at least one fluid unit and the at least one motion unit of the rolling membrane guiding catheter and/or the at least one device for synchronized operation. The automatic coupling can at least in part based on the motion state and/or the fluid state of the rolling membrane guiding catheter and/or the at least one device.

In an example, the computer program can cause the catheter system to execute any of the modes and/or any steps of the modes described herein. Any feature described in relation to modes and/or features of the catheter system can also be implemented in terms of instructions of a computer program and/or steps of a method and vice versa.

In a further example, the computer program can include further instructions. These may, when the program is executed, cause at least one motion unit and/or at least one fluid unit to determine the fluid state and/or the motion state of the rolling membrane guiding catheter and/or the at least one device such that a predetermined function is executed. This can relate to any of the predetermined functions, modes, and/or functionalities described herein. This yields the advantage of further automizing the according procedures to increase reliability, accuracy, treatment speed, and safety.

In some examples, the computer program can further include an interface for receiving image data of the treatment site. It can select the at least one control parameter at least in part based on the image data, wherein the selecting is at least in part based on a model that has been trained with a plurality of training data sets. Each training data set can include image data of a treatment site, at least one control parameter associated with the respective catheter operation, and optionally a performance value. This can be an advantageous application of artificial intelligence, e.g., machine learning, to select parameters for the treatment based on connections between information available on the patient and the planned treatment that a health professional might not be able to understand.

In an example, a computer program to induce a chiseling motion of the guidewire can be provided, further including an interface for receiving image data of the stenosis and selecting the at least one control parameter at least in part based on the image data. Selecting at least one control parameter in an automated way can improve safety, the suitability of the at least one control parameter, and reduce the risk of erroneous image evaluation by the health professional operating the rolling membrane catheter.

Such image data can be evaluated, e.g., by comparison with images of previously treated stenoses, to adjust treatment parameters associated with the chiseling motion, e.g., frequency, amplitude, relative position to the stenosis, and/or the used catheter, for example at least partially to the position, shape, and/or texture of the stenosis.

An exemplary computer program can further base the selecting of parameters at least in part on a (machine learning) model that has been trained with a plurality of training data sets, each training data set including image data of a stenosis, at least one control parameter which has been used for removing the stenosis, and optionally a performance value. Analogously, an according computer program can be provided to provide at least one parameter associated with any other action performed by the catheter.

The evaluation of the image data of a stenosis and the respective parameter selection can be based on training via machine learning, e.g., techniques like linear regression, logistic regression, decision tree, support vector machine (SVM) algorithm, naive Bayes algorithm, k-nearest neighbors algorithm, K-means, random forest algorithm, dimensionality reduction algorithms, gradient boosting algorithm, and Adaptive Boosting algorithm. This can facilitate the choice of suitable treatment parameters without the need for time-consuming and potentially error-prone image evaluation procedures.

Another aspect is a computer-implemented method for training an artificial intelligence model for predicting at least one control parameter for treating a stenosis (e.g. as outlined herein). The method can include: Inputting a plurality of training data sets to the model to train the model, each training data set including image data of a stenosis, at least one control parameter which has been used for removing the stenosis, and optionally a performance value.

The model can be based on techniques such as linear regression, logistic regression, decision tree, support vector machine (SVM) algorithm, naive Bayes algorithm, KNN algorithm, K-means, random forest algorithm, dimensionality reduction algorithms, gradient boosting algorithm, and AdaBoosting algorithm.

Another aspect is a computer-implemented method of predicting at least one control parameter for treating a stenosis (e.g. as outlined herein). The method can include: Predicting the at least one control parameter based on image data of the stenosis using an artificial intelligence model that has been trained as outlined herein.

Another aspect can be training data for training an artificial intelligence model for predicting at least one control parameter for treating a stenosis. The training data can include a plurality of training data sets, each training data set including image data of a stenosis, at least one control parameter which has been used for removing the stenosis, and optionally a performance value. Another aspect relates to a use of such training data for training an artificial intelligence model for predicting at least one control parameter for treating a stenosis.

The computer programs as outlined herein can be executed by a corresponding catheter having a processing unit, e.g. a microcontroller, a microprocessor, etc. In other examples, an external processing unit can execute the program to which the catheter as outlined herein can be coupled, e.g. wirelessly and/or in a wired manner.

It is noted that all functions described herein can be implemented as a corresponding functionality of the apparatus described herein, as a corresponding step of the methods outlined herein and/or as a corresponding instruction of the computer programs outlined herein. Even if described with reference to an apparatus, method and/or computer program, the aspects outlined herein can be applied to the respective other of an apparatus, method and/or computer program.

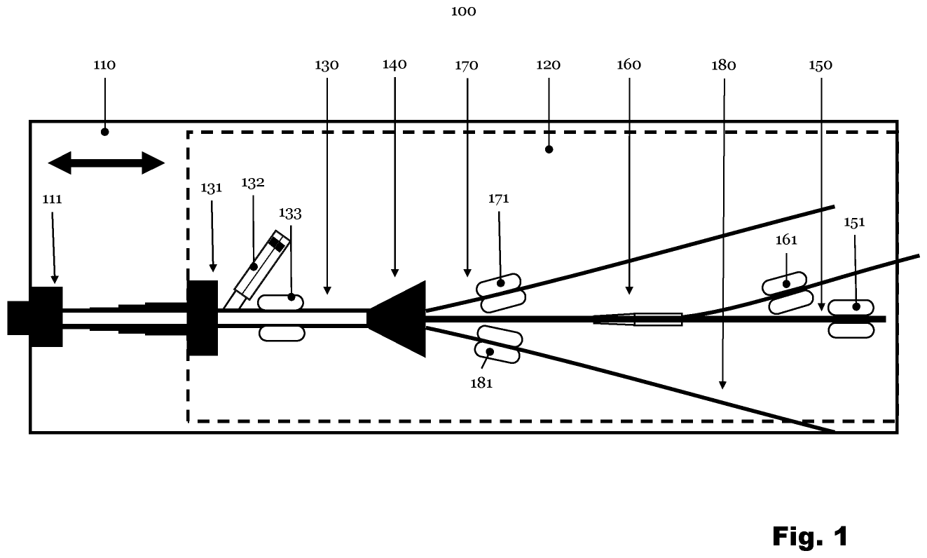

FIG. 1 shows an exemplary embodiment of a catheter system 100. The exemplary catheter system 100 includes a primary platform 110 including an introducer sheath 111. It further includes a secondary platform 120, wherein the secondary platform 120 is configured to move relative to the primary platform 110, such that the members of the secondary platform 120 can move relative to the introducer sheath 111, and thus can be inserted into the patient.

A telescopic connection element 131 can be provided to allow for a sealed insertion into the patient irrespective of the relative distance between primary and secondary platforms 110, 120. The connection element 131 can be adapted as a motion unit that can induce, e.g., when the guiding catheter is to be introduced into the blood vessel to be treated, the respective relative motion. However, in other examples, connection element 131 can be implemented as a passive element that adapts based on the relative motion that can be driven by a separate motion unit.

The catheter system 100 of FIG. 1 includes a rolling membrane catheter as a guiding catheter 130. The guiding membrane catheter 130 can be controlled by several components: The motion unit 131 can control the motion state of the outer shaft of the rolling membrane guiding catheter 130, the component for connecting to a fluid unit 132 (e.g. a valve, opening, etc.) controls the fluid state, e.g., providing a fluid pressure to the rolling membrane, and a further motion unit 133 controls the motion state of an inner shaft of the rolling membrane guiding catheter 130. In the exemplary embodiment, the inner shaft of the rolling membrane guiding catheter 130 can be operably coupled to docking unit 140.

Further members can be introduced into the guiding catheter 130 via the docking unit 140. First, a guidewire 150 controlled by a guide wire motion unit 151 can guide the guiding catheter 130 as well as other over-the-wire members, like in this exemplary embodiment a dilator catheter 160 controlled by a dilator motion unit 161. Further, the exemplary embodiment of FIG. 1 shows further exemplary catheters and 170, 180 which are controlled by their respective motion units 171, 181. The further catheters 170, 180 can be introduced in parallel to and independently from the guidewire 150. All the different catheters 130, 160, 170, 180 and the guidewire 150 can be controlled independently from each other and/or in a synchronized way via an external or internal control unit not shown in FIG. 1. Generally, all motion units 133, 151, 161, 171, 181 of the exemplary embodiment of FIG. 1 can include belts or wheels via which the motion state of the respective member is determined. Used belts or wheels can be easily replaced by new sterile belts, respectively, wheels.

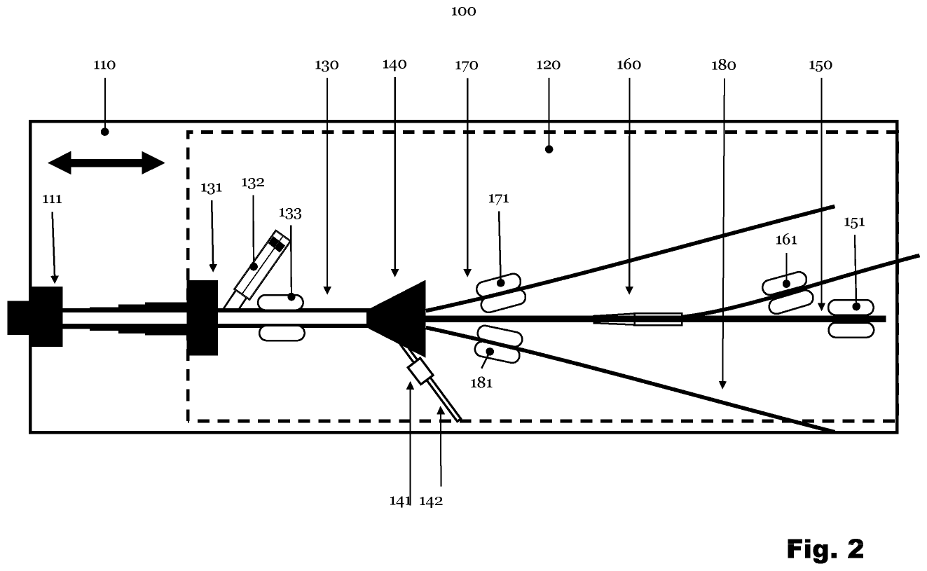

FIG. 2 shows a further exemplary embodiment of the catheter system 100. In the example of FIG. 2, the docking unit 140 includes a further port for fluidly connecting a supply line 141 with the inner shaft of the rolling membrane guiding catheter 130. The supply line 141 is configured to carry, for example, a contrast agent. The supply line 141 includes a valve 142 for (automized) opening, respectively, closing of the supply line 141.

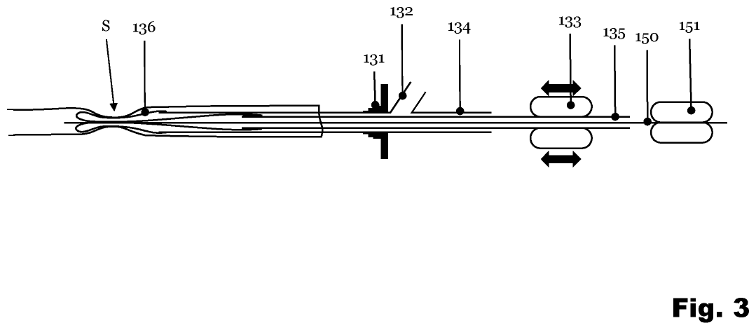

FIG. 3 shows an exemplary embodiment of a guiding catheter 130. In the example of FIG. 3, the guiding catheter 130 is a rolling membrane catheter with a rolling membrane 136 which is connected to a distal end of an inner shaft 135 and a distal end of an outer shaft 134. The exemplary guiding catheter 130 includes a guidewire 150 controlled by a motion unit 151. The motion unit 151 can induce axial translation as well as rotation around the length axis to advance the guidewire 150 through a lumen to and/or beyond the treatment site. The introduction of any member of the guiding catheter 130 must therefore follow the path of the guidewire 150. The guiding catheter 130 is introduced into the blood vessel via the motion unit 131 which drives the outer shaft 134 in a distal direction. In FIG. 3, the rolling membrane 136 is partly everted through a blood vessel and through a stenosis S. The fluid unit 132 provides a sufficient pressure to the volume between the inner shaft 135 and the outer shaft 134 which is limited at the distal end by the rolling membrane 136. This keeps the rolling membrane 136 in an inflated state such that its eversion can occur in a rolling motion when the inner shaft 135 moves distally relative to the outer shaft 134. The motion of the inner shaft 135 is induced by the motion unit 133. The pressure provided to the rolling membrane 136 can provide sufficient pressure to the stenosis S via the inflated rolling membrane to widen the stenosis. Further components like stents, balloons, etc. can be introduced to the site of the at least partly widened stenosis S to further widen the stenosis S. The rolling membrane 136 can be retracted in the reverse motion wherein the rolling membrane 136 is in an at least partly inflated state and the inner shaft 135 is moved proximally by the motion unit 133. As the guidewire is retracted with the same speed as the inner shaft, the procedure can be interrupted when having retracted the distal tip of the rolling membrane for a few millimeters. The rolling membrane can be temporarily depressurized to be able to push the guidewire back into its original position by the guidewire belts.

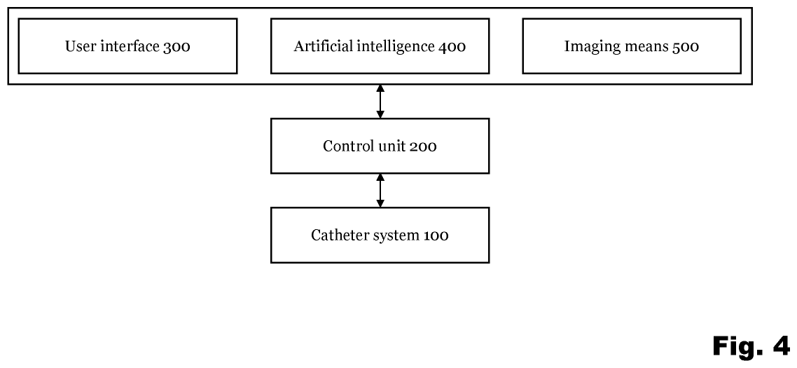

FIG. 4 shows a schematic representation of an exemplary configuration of catheter system 100 within an operating environment. The operating environment can include a control unit 200, a user interface 300, an artificial intelligence 400, and an imaging device 500. Therein, the user interface 300, an optional artificial intelligence 400, and an optional imaging device 500 can be configured to exchange signaling between each other to provide a common output provided to the control unit 200 and to receive input from the control unit 200. The control unit can then, based at least in part on the output, send signaling to the catheter system 100 and/or receive signaling from the catheter system 100 as described herein. Any other configuration is also possible, in which any of these building blocks is not included or replaced by other components and/or wherein the signaling pathways between the shown building blocks are altered, e.g., in terms of direction and which building blocks communicate directly.

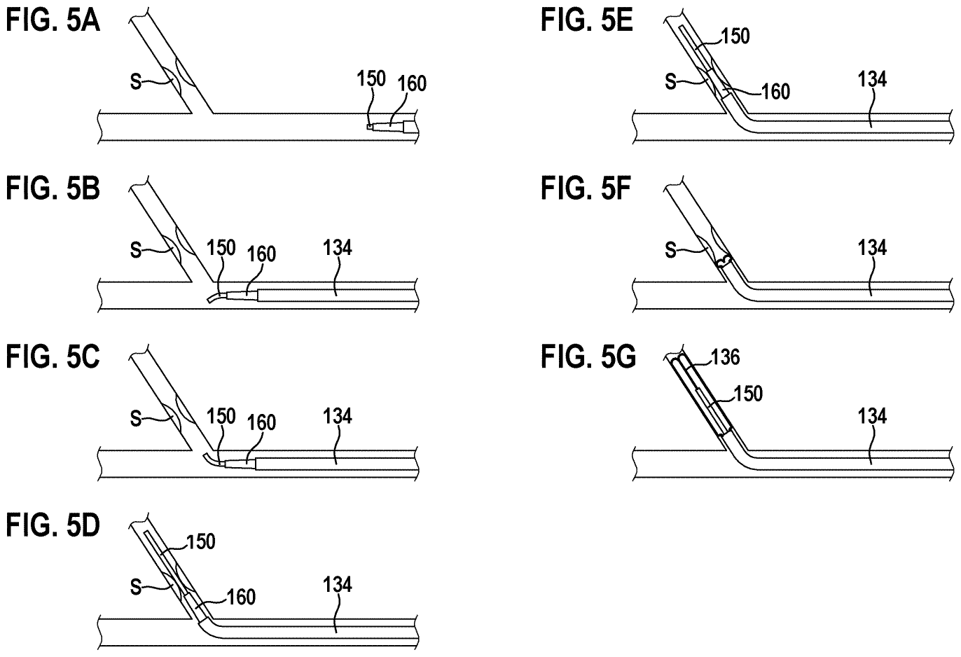

FIGS. 5A-5G show multiple schematic representations of snapshots an exemplary treatment of a stenosis S by a catheter system according to FIG. 1 including the rolling membrane guiding catheter 130 of FIG. 3, a dilator catheter 160, and a guidewire 150.

FIG. 5A shows a schematic representation of introduction of the rolling membrane guiding catheter 130 into a cardiovascular system. This can be induced by the at least one motion unit 131 (not shown in FIG. 5A).

FIG. 5B shows a schematic representation of advancement of the rolling membrane guiding catheter 130 into cardiovascular system op to a position at which the blood vessel splits into two canals. The flexible tip of the guidewire 150 is pushed outside the dilator catheter 160 by the belts 151 as far as needed to engage the bifurcation.

FIG. 5C shows a schematic representation of a guidewire 150 rotation to point the bent tip of the guidewire 150 into the direction of the treatment site, the stenosis S. This can by induced by the at least one guidewire motion unit 151 (not shown in FIG. 5C). This prepares the intended advancement of the guidewire 150 to lead the catheter 130 to be guided to the stenosis S. The dilator catheter 160 continuously builds up the bending moment from the soft guidewire 150 to the stiffer distal end of the guiding catheter 130 in the not rolled out state so that the guidewire 150 is enabled to guide the guiding catheter 130.

FIG. 5D shows a schematic representation of the advancement of the rolling membrane guiding catheter 130 and the guidewire 150 to and beyond the treatment site. In its advancement, the rolling membrane guiding catheter 130 follows the guidewire 150 which can be controlled in its advancement by rotating the bent tip accordingly.

FIG. 5E shows a schematic representation of the dilation of an exemplary stenosis S by a dilator catheter 160. The dilator catheter 160 has a conical tip to build up the bending moment as explained before and can also be configured to widen the stenosis S when the tip is advanced to and beyond the stenosis. In the exemplary treatment process, the dilation by the dilator catheter can serve as a pre-dilation preparing the following steps (a pre-dilation of the dilator catheter is optional).

FIG. 5F shows a schematic representation of the retracted dilator catheter 160 and guidewire 150. They can both be retracted into the rolling membrane guiding catheter 130 or fully removed through the docking unit 140 (not shown in FIG. 5F). The guidewire can also be left inside the stenosis for the next step if it has already crossed the lesion.

FIG. 5G shows a schematic representation of the eversion of the rolling membrane 136 of the rolling membrane guiding catheter 130 to widen the stenosis S. The fluid unit 132 (not shown in FIG. 5F) can provide the fluid pressure to the rolling membrane volume to evert the rolling membrane 136 to apply pressure to and widen the stenosis S. The catheter system 100 can be removed after this final step or further measures can be taken like, e.g., placing a stent.

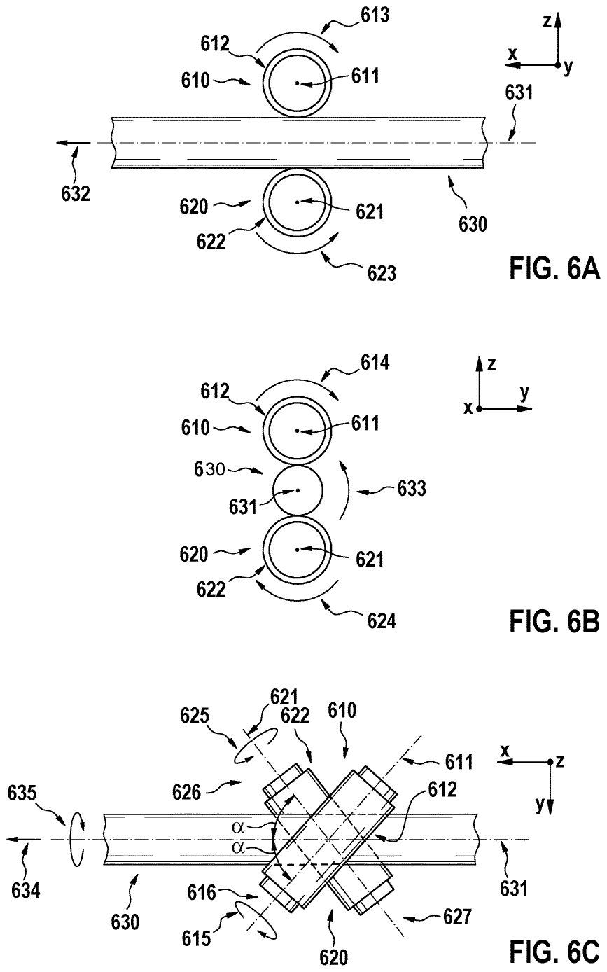

FIG. 6A-6C show different arrangements of a first transport roller 610 and a second transport roller 620. The first transport roller 610 and the second transport roller 620 are part of a motion unit for controlling the motion state of a catheter 630 including a length axis 631. The first transport roller 610 can rotate clockwise and counterclockwise around a first rotation axis 611. The second transport roller 620 can rotate clockwise and counterclockwise around a second rotation axis 621. The first and the second transport roller 610, 620 include a silicone sheathing 612, 622 each, which increases the friction between the catheter 630 and the first and second transport roller 610, 620 as well as protects the catheter 630 from damage and contamination. The silicone sheathings 612, 622 are detachably connected to the first, respectively, second transport roller 610, 620, so that the silicone sheathings 612, 622 can be slipped on and off from the transport roller.

The first and the second transport roller 610, 620 can be moved along the z-axis to form a gap in between the first and the second transport roller 610, 620 into which the catheter 630 fits in a force-fitting manner. Further, the first and the second transport roller 610, 620 can be rotated around an axis parallel to the z-axis.

FIG. 6A shows a cross-sectional view of a first arrangement. The first transport roller 610 rotates clockwise around the first rotation axis 611 (as indicated by arrow 613) which is parallel to the y-axis. The second transport roller 620 rotates counterclockwise around the second rotation axis 621 (as indicated by arrow 623) which is parallel to the y-axis. Thus, the catheter 630 is translationally moved along its length axis 631 as indicated by arrow 632.

FIG. 6B shows a cross-sectional view of a second arrangement. The first transport roller 610 rotates clockwise around the first rotation axis 611 (as indicated by arrow 614) which is parallel to the x-axis. The second transport roller 620 rotates clockwise around the second rotation axis 621 (as indicated by arrow 624) which is parallel to the x-axis. Thus, the catheter 630 is rotated around its length axis 631 as indicated by arrow 633.

FIG. 6C shows a top view of a third arrangement. The first rotational axis 611 and the length axis 631 of the catheter 630 enclose an angle a in the x-y-plane, whereas the second rotational axis 621 and the length axis 631 of the catheter 630 enclose an angle −α in the x-y-plane. In other words, the projection of the first rotational axis 611 into the x-y-plane is mirror-symmetrical to the projection of the second rotational axis 621 into the x-y-plane with respect to the length axis 631 of the catheter 603. The first rotational axis 611 rotates as indicated by arrow 615. The second rotational axis 621 rotates as indicated by arrow 625. Accordingly, the left end of the first transport roller 616 rotates in the same direction as the left end of the second transport roller 626, whereas the left end of the first transport roller 616 rotates in the opposite direction to the right end of the second transport roller 627. Thus, the catheter 630 is translationally moved along its length axis 631 as indicated by arrow 634 and rotated around its length axis 631 as indicated by arrow 635.

While specific embodiments of the present invention have been shown and described, it should be understood that other modifications, substitutions and alternatives are apparent to one of ordinary skill in the art. Such modifications, substitutions and alternatives can be made without departing from the spirit and scope of the invention, which should be determined from the appended claims.

Various features of the invention are set forth in the appended claims

1. A catheter system comprising:
a rolling membrane guiding catheter;
a docking unit configured to automatically insert a plurality of devices into the rolling membrane guiding catheter.

2. The catheter system of claim 1, wherein the plurality of devices comprises at least one of the following: a guidewire, a dilator catheter, a balloon catheter, a stent, an additional rolling membrane catheter, a biopsy tool, an endoscope, an intravascular ultrasound, (IVUS) tool, a fractional flow reserve, (FFR) tool and/or an optical coherence tomography, (OCT) tool.

3. The catheter system of claim 1, claim 1, comprising at least one fluid unit configured to determine a fluid state of a rolling membrane of the rolling membrane guiding catheter and/or at least one of the plurality of devices.

4. The catheter system of claim 4, comprising at least one motion unit configured to determine a motion state of the rolling membrane guiding catheter, a rolling membrane of the rolling membrane guiding catheter, and/or at least one of the plurality of devices.

5. The catheter system of claim 4, wherein the at least one motion unit is configured to determine a translational component of the motion state and/or a rotational component of the motion state.

6. The catheter system of claim 4, wherein the at least one motion unit is configured to be provided with an exchangeable and/or disposable transport belt.

7. The catheter system of claim 1, comprising means for identifying at least one of the plurality of devices.

8. The catheter system of claim 1, wherein the docking unit comprises a pressure-tight seal.

9. The catheter system of claim 1, comprising imaging means for providing image data and/or means for image evaluation of image data.

10. The catheter system of claim 9, configured to determine the fluid and/or motion state at least in part based on the image data.

11. The catheter system of claim 1, comprising a control unit operably coupled to the catheter system and configured to provide automatic and/or synchronized control of at least one parameter associated with a motion state and/or a fluid state of the rolling membrane guiding catheter and/or of at least one of the plurality of devices.

12. The catheter system of claim 11, comprising a user interface, wherein the control unit is configured to control the at least one parameter at least in part based on the user input.

13. The catheter system of claim 1, comprising a primary platform and a secondary platform, wherein the primary platform comprises an introducer sheath into the patient, wherein the secondary platform comprises the docking unit and is configured to be movable relative to the primary platform.

14. A computer program comprising instructions which, when the program is executed, causes the catheter system of claim 4 to automatically couple the at least one fluid unit and the at least one motion unit and/or the at least one device for synchronized operation.

15. The computer program of claim 14, comprising instructions which, when the program is executed, causes the at least one motion unit and/or the at least one fluid unit to determine the fluid state and/or the motion state of the rolling membrane guiding catheter and/or the at least one device such that at least one of a plurality of predetermined functions is executed.

16. The catheter system of claim 1, comprising at least one motion unit configured to determine a motion state of the rolling membrane guiding catheter, a rolling membrane of the rolling membrane guiding catheter, and/or at least one of the plurality of devices.
